(19) United States
(12) Patent Application Publication
(10) Pub. No.: US 2019/0380741 A1
(43) Pub. Date: Dec. 19, 2019

(54) BIOPSY NEEDLE GUIDE WITH TWO-STAGE FIXING

(71) Applicant: Hubert Noras, Würzburg (DE)

(72) Inventor: Hubert Noras, Würzburg (DE)

(21) Appl. No.: 16/489,874

(22) PCT Filed: Feb. 9, 2018
(86) PCT No.: PCT/DE2018/100111

§ 371 Date: Aug. 29, 2019

(30) Foreign Application Priority Data
Mar. 1, 2017 (DE) 10 2017 104 301.2

(51) Int. Cl. A61B 17/34; A61B 10/02

(52) U.S. Cl. CPC A61B 17/3403; A61B 2017/00486; A61B 2017/3407; A61B 10/0233

(57) ABSTRACT

A biopsy needle guide, which has at least two degrees of freedom and can be freely positioned at least in a plane, includes a through-passing opening for passing a biopsy needle through. In an interior of the opening, a fixing structure and a clamping structure are present, and an adapter sleeve is present, which has a continuous opening for passing a biopsy needle through, as well as a stop collar, and which can be pushed into the opening as far as the collar and is rotatable after pushing in as far as the stop. By rotating the adapter sleeve to a first angle at most, a latch structure of the adapter sleeve engages behind the fixing structure of the opening, so that the adapter sleeve is fixed in the axial direction. By rotating the adapter sleeve beyond the first angle, the latch structure interacts with the clamp structure such that a biopsy needle that is introduced in the opening is fixed in the axial direction.

### CROSS-REFERENCE TO RELATED APPLICATIONS

This Application is a Section 371 National Stage Application of International Application No. PCT/DE2018/100111, filed Feb. 9, 2018, the content of which is incorporated herein by reference in its entirety, and published as WO 2018/157882 on Sep. 7, 2018, not in English.

### FIELD

Embodiments of the present disclosure relate to a biopsy needle, guide, comprising a head, which has at least two degrees of freedom and can be freely positioned, at least in a plane, and has a through-passing opening (20) for passing through a biopsy needle, as well as a use of such a biopsy needle guide.

### BACKGROUND

By the term biopsy is meant the taking and examination of tissue samples from a living organism. A biopsy is often advisable in particular after diagnosis of a tumour, in order to directly examine the tumour tissue medically and, for example, make a decision about the malignancy and benignancy of the tumour.

A biopsy is usually performed by puncture of the target tissue with a biopsy needle. This comprises introducing the biopsy needle and positioning the needle tip in the tissue to be examined with subsequent taking of tissue samples.

A problem with this results when the tissue to be biopsied lies in the interior of the body and is not visible from the outside. In this case, a reliable biopsy of a relatively small target volume is only possible by means of a common insertion of a sufficiently high-resolution imaging method together with a biopsy needle guide is possible, which, by means of the images of the tissue to be sampled produced by means of the imaging method, permits the needle, or the needle tip, to be introduced in a targeted manner into the tissue.

Biopsy needle guides usually achieve this by the fact that a head, comprising an opening for passing through the biopsy needle, is guided such that it is freely movable in a plane via at least two degree of freedom. If the position of the head is known relative to the tissue to be examined, either in that the head as a whole, or at least a portion thereof, is also imaged by means of the imaging method, then, by moving the head or the opening for the biopsy needle that is present therein, it can be passed precisely over the position to be sampled, such that a needle pushed through the opening must always hit the correct position. For a successful biopsy, however, it is further necessary that the insertion depth of the needle is known with sufficient accuracy. This can be achieved in that, by imaging in a plane that is tilted with respect to the first plane, the depth information is determined in the form of the distance from the head to the sampled point or before positioning of the head, the distance between the movement plane of the head to the first image plane is determined. After positioning of the head, a biopsy needle is then passed through the needle guide opening of the head accurately by the determined distance over that reference point of the needle guide or of the head that is crucial for distance determination, thus positioning the needle tip in the target volume to be sampled.

After removal of the sample, the biopsy needle is extracted again. Here, it is almost unavoidable that a contamination of the needle guide or at least the head tissue material of the patient takes place. For reliable and hygienic reuse of the needle guide, it would thus be necessary to sterilize the head between two biopsy operations. This is difficult to perform with the required hygiene, due to the form being technically complex for technical reasons and the multi-part structure of the head, and is in any case associated with a high time consumption and great expense.

As a solution to this problem, it is know not to bring the biopsy needle guide into direct contact with the head, but to use a so-called adapter sleeve, which is inserted into the guide opening of the head, the adapter sleeve itself having in turn a continuous opening, which serves to receive and guide the biopsy needle. After introduction of the needle tip into the desired target volume, an axial fixing of the needle is necessary before or for removal of the tissue sample. To this end, a clamping device can be provided, for example in the passage opening of the head of the biopsy needle guide.

With the use of an adapter sleeve, however, the problem arises that, due to a clamping device in the head itself, only the adapter sleeve, but not the needle, is clamped. A possible solution would be to design the sleeve to be correspondingly thin walled, so that a clamping by means of a clamping device integrated in the head acts on the biopsy needle through the sleeve. However, this has the disadvantage, first, that a sufficiently large separation between the contaminated biopsy needle and the biopsy needle guide head is no longer ensured, or the mechanical forces necessary for fixing would be unacceptably high.

However, the unavoidable disadvantage of this is that the biopsy needle and adapter sleeve would be simultaneously axially fixed. This means that before fixing of the biopsy needle, the adapter sleeve would also remain movable in the axial direction, which, first, makes the exact positioning of the biopsy needle more difficult and also that, on extraction of the needle, it would be made necessary that the sample remover actively, for example by holding with one hand, prevents the adapter sleeve from sliding out prematurely.

### SUMMARY

Against the above-background, it is an object of the present disclosure to provide a biopsy needle guide with sufficient separation between needle and parts of the needle guide that are not easily sterilizable, which permits a clamping of the needle with little force application and operation that is as simple and ergonomic as possible.

As a solution, embodiments of the present disclosure include a biopsy needle guide with a head, which has an opening to receive an adapter sleeve, the adapter sleeve comprising in turn an opening for passing a biopsy needle through, the opening, in the head of the needle guide, which in the interior is equipped with a fixing and a clamping structure, which interacts with a latch structure or section of the adapter sleeve, so that a two-stage fixing of adapter sleeve and biopsy needle is achieved.

Herein, the latch section of the adapter sleeve, on rotation of the adapter sleeve about its longitudinal axis up to a first angle, engages behind only the fixing structure, so that a fixing of the adapter sleeve in the axial direction, but not yet any clamping of the biopsy needle, takes place. On rotation beyond the first angle, the latch section furthermore engages behind the clamping structure, and a clamping of the biopsy needle is achieved. By this means, a type of two-stage bayonet lock is implemented, which in the first stage only effects the axial fixing of the adapter sleeve, the biopsy needle remaining freely movable in order to clamp this, too, in the second stage. The possibilities for achieving this basic concept of the present disclosure, however, are not limited to the known embodiments of a bayonet lock with tongue and groove, but more general embodiments are also conceivable.

One advantage of this solution is a lightweight and ergonomically operable axial fixing, which permits a precise movement in the function of the patient. After the needle has been fully positioned, a slight rotation beyond the first angle is enough to also . . . the needle itself. the sterility of the needle guide head or of the needle guide as a whole is ensured by the use of an adapter sleeve, which is either easily sterilizable or, if a particularly high degree of hygiene appears necessary, can also be used as a single-use part. After removal of the tissue sample, the sample remover, on extraction of the biopsy needle need not worry that the adapter needle is released too early from its biopsy needle guide head, since, as long as it remains rotated by an angle lying between zero degrees and the first angle, with respect to its insertion position, is not axially movable. As a result, one-hand operation of the biopsy needle guidance is advantageously permitted.

The use of the biopsy needle guide according to embodiments of the present disclosure provide that, first the head is displaced to an optimum position with the aid of the determined image data, any rotational or pivoting degrees of freedom of the head possibly also being taken into account, in order to obtain an insertion direction (angulation) that is better for the patient, the adapter sleeve is then introduced into the opening and, by rotation about an angle that is smaller than the first angle, is fixed in the axial direction, the biopsy needle is then inserted through the opening in the adapter sleeve and into the tissue in the patient, which is to be sampled. Finally, when the end position of the needle is reached, the needle and sleeve are both clamped by further rotation of the adapter sleeve beyond the first angle.

An advantageous embodiment of the present disclosure, which can be realized individually or in combination, in so far as they do not obviously preclude one another, are presented below.

The adapter sleeve according to embodiments of the present disclosure comprises, first, a body that is designed approximately as a hollow cylindrical shank. At one end of this shaft a stop collar is disposed behind the handle in the insertion direction in the form of two or more grip tabs. The latch section of the adapter sleeve is preferably a part of the shank. In one embodiment, it consists of a tongue, which is cut out of the material on three sides and of which the loose end is movable essentially in the radial direction. In another embodiment, the latch section consists of a latch pin, which is inserted into an opening, which breaks through the circumferential surface of the adapter sleeve, and is movable in the radial direction.

The fixing and clamping structure on the interior surface of the passage opening of the head can, in one embodiment, consist of a groove which, in a first section, has a constant radius and in a second section, which forms the clamping structure, has a radius, which visibly reduces with an increasing angle.

In another embodiment, the fixing structure consists of a bridge, which projects into the essentially circular passage opening. In one variant, this comprises a first section, which serves for axial fixing of the adapter sleeve without clamping, and a second section, which, together with the latch section, effects a clamping of both the biopsy needle and adapter sleeve.

In a further variant, the bridge only represents the fixing structure and the clamping structure is formed by a further section of the inner surface of the needle passage structure, which is different from the bridge and has a radius which is reduced compared to an average radius of the opening.

In some embodiments, a plurality of latch parts in the adapter sleeve or, corresponding thereto, a plurality of fixing and clamping structures are present in the passage opening of the head. The passage opening of the head preferably has a circular, circular-segmental or a circular sector-shaped cross-section and, in the case of a latch section in the adapter sleeve, which projects beyond the average radius, has guide grooves, which permit a frictionless introduction of the adapter sleeve together with the latch section until it meets the stop.

The head of the biopsy needle guide according to embodiments of the disclosure may be fixed via a clamping screw on a guide carrier, on which it is movably guided, and particularly preferably designed such that it can be angulated, that is to say it is pivotable about an axis which lies within the movement plane of the head. Further preferably, the head can be clamped in discrete steps, also when angulated. It also has, in addition to the passage opening, a receiver for a marking element, which permits, for example when CT or MRT is used as an imaging method, the exact position of the head on the image of the tissue that is to be sampled to be recognised.

In some embodiments, for more ergonomic operation, the adapter sleeve has a handle part in the form of two or a plurality of grip tabs.

In an additional step of the use of the biopsy needle guide according to embodiments of the present disclosure, by reverse rotation of the adapter sleeve, behind the first angle the clamping of the biopsy needle is preferably released, so that it is movable in the axial direction, for example, in order to extract it from the tissue again, without an axial displacement of the adapter sleeve taking place, because this continues to be displaced by interaction of the latch section of the adapter sleeve with the fixing structure in the interior of the passage opening of the head.

Further details and features are described below with reference to exemplary embodiments which are described in greater detail by means of the illustration. These are only intended to illustrate embodiments of the present disclosure, and in no way to limit its generality.

### BRIEF DESCRIPTION OF THE DRAWINGS

FIG. 1 shows an embodiment of a needle guide head of the present disclosure with partly inserted adapter sleeve.

FIG. 2 shows an embodiment of a biopsy needle guide according to the present disclosure with the head from FIG. 1.

FIG. 3 shows possible embodiments of the passage opening of the head with clamping and fixing structures.

### DETAILED DESCRIPTION OF ILLUSTRATIVE EMBODIMENTS

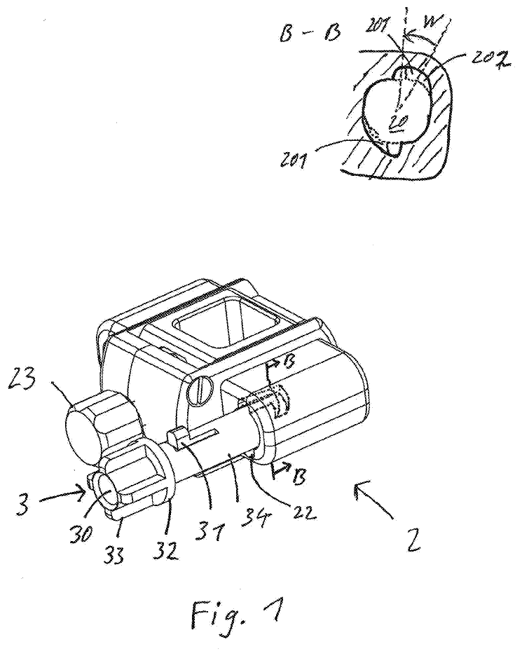

FIG. 1 shows, in perspective view, a possible embodiment of a head of a biopsy needle guide according to embodiments of the present disclosure, as well as a cross-section through the part of the head that contains the passage opening.

Head 2 can be seen, in the passage opening of which, to receive a biopsy needle, adapter sleeve 3 is partly inserted.

Adapter sleeve 3 comprises a hollow-cylindrical shank 34 on the upper end of which there is disposed a stop collar having, behind it, a grip part, in the form of four grip tabs 33 which are arranged offset by 90° with respect to one another. In an opening breaking through the shank, a latch pin 31, which projects beyond the upper circumferential surface of the shank, is mounted such that it is radially movable. Opening 20 in head 2 has grooves 22 for introducing the latch pin 31 as far as the stop, which is implemented by striking of the stop collar 32 against the end face of that partial section of head 2 that contains the passage opening. After the complete insertion of the adapter sleeve 3 as far as stop 32, said adapter sleeve is rotatable about a particular angle due to the presence of a tangential groove. On rotation, latch pin 31 engages behind the lateral surface of the tangential groove, the adapter sleeve being fixed against axial movement in the manner of a bayonet lock. The tangential groove has, as becomes clearer in section B-B, a first section 201, in which the effective radius remains essentially constant, so that, on rotation up to a first angle, only an axle fixing of the adapter sleeve, not however a clamping of the biopsy needle guided by the adapter sleeve. Beyond the first section 201, a second section 202, in which the effective radius of the tangential groove is reduced, which on further rotation of the adapter sleeve beyond the first angle, latch pin 31 is pushed inward radially into opening 30 of the adapter sleeve, a clamping of a biopsy needle present therein being achieved. Thus a type of two-stage bayonet lock is realized, which, in the first stage only results in axial fixing with free movability of the biopsy needle and in the second stage achieves complete clamping both of an adapter sleeve and biopsy needle.

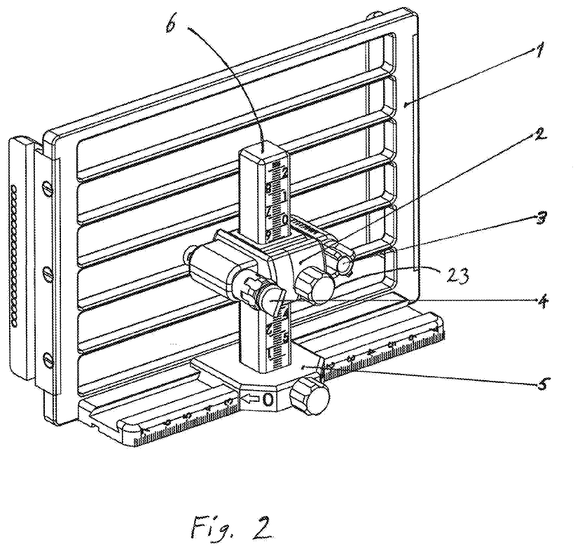

FIG. 2 shows in perspective view an embodiment of a biopsy needle guide according to embodiments of the present disclosure with the head from FIG. 1. Head 2 is mounted on guide carrier 5 such that it can be displaced and can be fixed there by means of clamping screw 23. Guide carrier 5 in turn is guided so as to be linearly movable on a guide rail 6. Two translation degrees of freedom are thus effectively available, with which the needle guide head, within a certain traverse range, is freely movable in the plane formed by guide carriers 5 and rail 6.

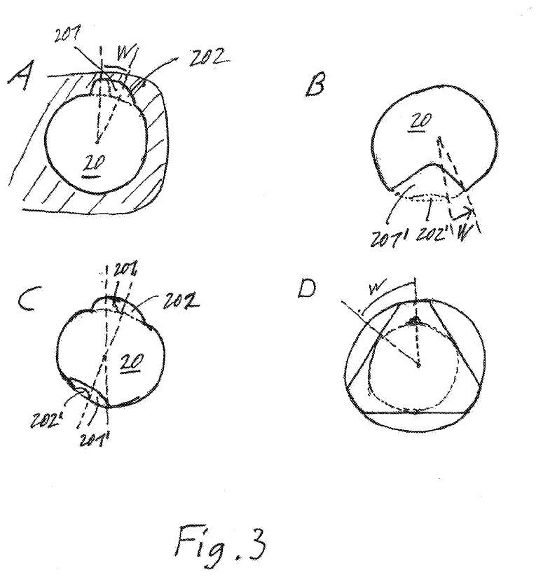

FIG. 3 shows different possible embodiments of fixing and clamping structures in opening 20 of the head 2.

Partial figure A generally shows the embodiment known from FIG. 1, though in this case a version with only one groove.

Partial figure B shows an embodiment in which the fixing structure consists of bridge 201, which projects into the essentially cylindrical opening 20. Bridge 201 has a coarsely triangular cross-section. The fixing here achieved by interaction with an azimuthally extending notch, which is inset into the shank of the adapter sleeve, which is not shown here. That part of the sleeve shank that lies behind bridge 201 in the insertion direction when the adapter sleeve is inserted must herein have a recess that, in cross-section, at least corresponds to the bridge or contains it, in order to permit an insertion of the sleeve. Clamping of the biopsy needle could be readily achieved by means of a latch pin which is positioned in the azimuthal notch of the adapter sleeve. The first angle herein would be then angular distance between the beginning of the notch and the latch pin. Here, however, an additional clamping structure in the form of a region 202 with a radius that is reduced in comparison to the radius of the opening 20.

Partial figure C shows an embodiment in which the fixing and clamping structures of partial figures A and B are combined.

Partial figure D presents a further possible embodiment of the clamping and latching structures. Here, they are in the form of three bridges which are in each case offset by 120 degrees.

The shank of an adapter sleeve that is compatible therewith has a cross-section, which, in shape, corresponds to that part of the opening 20 that is left exposed by the bridges and, in each of the triangular arms, has an azimuthal notch that cooperates with the bridges and which extends downward to a depth that corresponds to the radius of the inner circle (dotted circle) of the exposed part. If the latch pin or pins are seated centrally in this notch, the indicated angular range results as the first angle W, within which only an axial fixing of the adapter sleeve, but not yet a clamping, takes place. If the sleeve is rotated further, the latch pin or pins are pressed against the bridges and by them pressed inward, where they clamp the biopsy needle.

### LIST OF REFERENCE CHARACTERS

1 Frame
2 Needle guide head
20 Needle guide opening
201 Tangential groove, first section
202 Tangential groove, second section
201 Bridge
202 Clamp region
22 Axial groove
3 Adapter sleeve
31 Latch pin
32 Stop collar
33 Grip lug
34 Shank
4 Marker pin
5 Carriages
6 Guide carriers

Although the embodiments of the present disclosure have been described with reference to preferred embodiments, workers skilled in the art will recognize that changes may be made in form and detail without departing from the spirit and scope of the present disclosure.

1. A biopsy needle guide, comprising
a head, which
has at least two degrees of freedom and can be freely positioned at least in a plane, and
has a through-passing opening for passing through a biopsy needle,
wherein:
in an interior of the opening, a fixing structure and a clamping structure are present,
an adapter sleeve is present, which has a continuous opening for passing through a biopsy needle as well as a stop collar, and which can be pushed into the opening as far as the collar and is rotatable after pushing in as far as the stop, and
by rotation of the adapter sleeve by a first angle at most, a latch structure od the adapter sleeve engages behind the fixing structure of the opening, so that the adapter sleeve is fixed in the axial direction, wherein a first rotation of the adapter sleeve beyond the first angle, the latch structure interacts with the clamp structure such that a biopsy needle that is introduced into the opening is fixed in the axial direction.

2. The biopsy needle according to claim 1, wherein the adapter sleeve comprises:
an approximately hollow cylindrical shank, which inserted into the opening of the head as the latch structure, a clamping pin which passes through the circumferential surface of the adapter sleeve and is mounted therein such that it is radially movable, and/or
grip tabs at a rear end.

3. The biopsy needle guide according to claim 1, wherein the fixing structure and the clamping structure are formed by
a groove, which is introduced into the inner surface of the opening and comprises a first section with a constant first radius for fixing without clamping and a second section with a radius, which tapers steadily from the first radius to a second radius, as clamping structure, or
a bridge, which projects into the essentially circular opening and comprises a section, which either serves as a fixing structure and a section which serves as clamping structure, or serves exclusively as fixing structure, the clamping structure being formed by a further section, which is present in the opening and has a radius which is reduced with respect to an average radius of the opening.

4. The biopsy needle guide according to claim 1, wherein the adapter sleeve comprises a plurality of the latch structures and the opening comprises a plurality of clamping and fixing structures.

5. The biopsy needle guide according to claim 1, wherein the opening of the head has a circular, circular segmental or circular section-shaped cross-section.

6. The biopsy needle guide according claim 1, wherein the opening of the head has guide grooves for the latch structure of the adapter sleeve.

7. The biopsy needle guide according to claim 1, wherein the head
is guided such that it is movable on a guide carrier, which is in turn mounted so as to be movable or rotatable and
can be fixed on the guide carrier by means of a clamping screw,
can be angulated, that is to say is pivotable with respect to the guide carrier about an axis, the head also being clampable when it is angulated, and/or
having a further opening for receiving a marker.

8. A method of using the biopsy needle guide according to claim 1, comprising:
moving the head to a location and orientation for the biopsy,
introducing the adapter sleeve into the opening of the head to the stop collar,
fixing the adapter sleeve in the axial direction by rotation through an angle that is smaller than or equal to the first angle,
guiding a biopsy needle through the opening of the adapter sleeve, and
fixing the biopsy needle against movement in the axial direction including rotating the adapter sleeve beyond the first angle.

9. The method according to claim 8, comprising:
releasing the fixing of the biopsy needle against movement in the axial direction including rotating back the adapter sleeve behind the first angle, and
extracting the biopsy needle.
